(19) United States
(12) Patent Application Publication
(10) Pub. No.: US 2017/0322210 A1
(43) Pub. Date: Nov. 9, 2017

(54) In Vitro Oral Biofilm Models of Interdental Spaces and Uses

(71) Applicant: Colgate-Palmolive Company, New York, NY (US)

(72) Inventors: Hans Stettler, Basel (CH); Amy Russo RUSSO, Belle Mead, NJ (US)

(73) Assignee: Colgate-Palmolive Company, New York, NY (US)

(21) Appl. No.: 15/523,792

(22) PCT Filed: Nov. 11, 2014
(86) PCT No.: PCT/US2014/065094

§ 371 Date: May 2, 2017

(51) Int. Cl. G01N 33/569; C12M 1/12; C12Q 1/18

(52) U.S. Cl. CPC G01N 33/56955; C12M 1/12; C12Q 1/18

(57) ABSTRACT

Disclosed is an in vitro oral biofilm model of interdental spaces including: a support, and a plurality of substratum pairs attached to the support, wherein an oral biofilm is capable of forming on the substratum pairs, wherein the substratum pairs each include a first member and a second member, and wherein the first member and the second member of each substratum pair are arranged to form a space interval between the first member and second member. Methods for assessing the formation of oral biofilms using the in vitro oral biofilm model and methods for testing agents, such as oral compositions, on biofilm reduction using the present model are also provided.

### BACKGROUND

Oral biofilms are matrix-enclosed microbial communities in which cells adhere to each other on the surfaces in the mouth. The colonization of clean oral surfaces by microorganisms (dental plaque) occurs within minutes and can lead to the development of two of the most common diseases in humans: dental caries or periodontitis, if left untreated.

Methods to inhibit biofilm growth on dental composites have been sought for several decades. Bacteria in biofilms are considerably more resistant to treatment with antimicrobials than their planktonic counterparts due to protective matrix secretions and species diversity. Several in vitro models exist for studying biofilms. For example, the Manchester Model is completely anaerobic and models sub-gingival plaque biofilms. The Academic Center for Dentistry in Amsterdam (ACTA) model, which was developed in 2010, models supra-gingival plaque biofilms and allows for active attachment of bacteria biofilms to glass disks rather than layers of sedimented cells. The ACTA model uses native saliva as the source of bacteria, and is relatively high throughput.

While these models may be useful for studying biofilm formation and testing the efficacy of compositions for biofilm reduction, neither the Manchester nor ACTA model can be used as a model for assessing biofilm formation in the spaces between teeth. Due to the large amounts of plaque which accumulate in this region, interdental spaces are particularly prone to caries and periodontal disease. Accordingly, there remains a need in the art for additional oral biofilm models including those which are capable of modeling the spacing between teeth.

### BRIEF SUMMARY

The present disclosure is directed to an in vitro oral biofilm model of interdental spaces including: a support, and a plurality of substratum pairs attached to the support, wherein an oral biofilm is capable of forming on the substratum pairs, wherein the substratum pairs each include a first member and a second member, and wherein the first member and the second member of each substratum pair are arranged to form a space interval between the first member and second member.

In another aspect, the present disclosure is directed to a method of forming an oral biofilm, the method including: providing a substratum pair attached to a support, wherein the substratum pair includes a first member and a second member, and wherein the first member and the second member are separated to form a space interval between the first member and the second member, and providing a liquid growth medium including microorganisms capable of oral biofilm production; placing the substratum pair into the liquid growth medium; and incubating the substratum pair in the liquid growth medium to form a biofilm on the substratum pair.

The present disclosure is also directed to a method for identifying an agent for reducing biofilm in interdental spaces, the method including: providing at least a first substratum pair and a second substratum pair attached to a support, wherein an oral biofilm is capable of forming on the substratum pairs, wherein each substratum pair includes a first member and a second member, and wherein the first member and the second member are separated to form a space interval between the first member and second member; providing a liquid growth medium comprising microorganisms capable of oral biofilm production; placing at least the first substratum pair and the second substratum pair into the liquid growth medium including microorganisms capable of oral biofilm production; incubating the at least first substratum pair and the second substratum pair, thereby forming a biofilm on at least the first substratum pair and the second substratum pair; contacting the first substratum pair with a test agent; and comparing an amount of biofilm on the first substratum pair with an amount on the second substratum pair; and indicating that the test agent reduces biofilm when the amount of biofilm on the first substratum pair is smaller in comparison to the amount of biofilm on the second substratum pair.

Further areas of applicability of the present disclosure will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Embodiments of the present disclosure will become more fully understood from the detailed description and the accompanying drawings, wherein:

FIG. 1 depicts examples of substratum pairs with 2× and 3× intervals between each member of a substratum pair and a single substratum (glass coverslip).

FIG. 2 depicts an embodiment of a plurality of substratum pairs with 2× intervals between each member of a substratum pair.

FIG. 3 depicts an embodiment of a plurality of substratum pairs with half of the substratum pairs having 2× intervals between the members and half of the substratum pairs having 3× intervals between the members.

FIG. 4 depicts the optical density of bacterial cells observed after five days of bacterial growth on two in vitro oral biofilm models having 2× or 3× intervals between each member of a substratum pair in comparison to the optical density of bacterial cells observed using an oral biofilm model with only a single glass disk substratum for an exemplary implementation.

FIG. 5 shows the percentage of dead bacterial cells observed after treatment with test agents (toothpaste alone versus toothpaste and mouthwash) on biofilms formed on in vitro oral biofilm models of interdental spaces and an oral biofilm model using a single glass slide for an exemplary implementation.

FIG. 6 shows the amount of resazurin fluorescence observed after treatment with test agents (toothpaste alone versus toothpaste and mouthwash) on biofilms formed on in vitro oral biofilm models of interdental spaces and an oral biofilm model using only a single glass slide for an exemplary implementation.

### DETAILED DESCRIPTION

The following description of the embodiments is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

The In Vitro Oral Biofilm Model of Interdental Spaces

The present disclosure relates to an in vitro oral biofilm model of interdental spaces, methods for assessing the formation of oral biofilms and methods for testing agents, such as oral compositions, on biofilm reduction.

As used herein, oral biofilms refer to three-dimensional structured bacterial communities which are embedded in an exo-polysaccharide matrix and attached to a solid surface.

The oral biofilm model of the present disclosure includes substratum pairs, which are attached to a support. As used herein, the term “substratum” refers to a natural or synthetic material upon which an oral biofilm may be formed. Examples of a natural substratum include enamel or hydroxyapatite. The natural specimens may be obtained from any mammal including but not limited to humans, non-human primates, camels, cats, chimpanzees, chinchillas, cows, dogs, goats, gorillas, horses, llamas, mice, pigs, murine, rats and sheep.

In some embodiments, the substrata include synthetic materials which are used to form dental implants, e.g., titanium, ceramics. Other synthetic materials which permit biofilm formation and which may be used for the substratum pairs of the oral biofilm model of the present disclosure include but are not limited to synthetic hydroxyapatite, glass, silicon, urethane, or similar materials.

The synthetic material substratum may be of any shape, such as a tooth shape, a rectangle, a square or a circle. For example, the substrata may be in the form of glass disks or hydroxyapatite disks.

The substrata may be in pairs to model interdental spaces. Each member of a substratum pair may be prepared from the same material or a different material. In some embodiments, each member of a substratum pair is prepared from the same material, e.g., each member is a glass disk.

In various embodiments, members of a substratum pair are arranged with respect to each other in manner that forms a constant or varying space interval between the members. Some embodiments may be arranged to include a space interval that varies from a distance ranging from 0.1 mm to up to 4 or more millimeters. In various embodiments, the first member and the second member of the substratum pair may be separated from each other by a separation element, which arranges the constant or varying space interval between the members. In various embodiments, the separation element may attach the first member and the second member of the substratum pair to each other. In some embodiments, the first member and the second member of the substratum pair may be attached to each other using any means known in the art, such as the use of adhesives including biocompatible adhesives, e.g., dental adhesives.

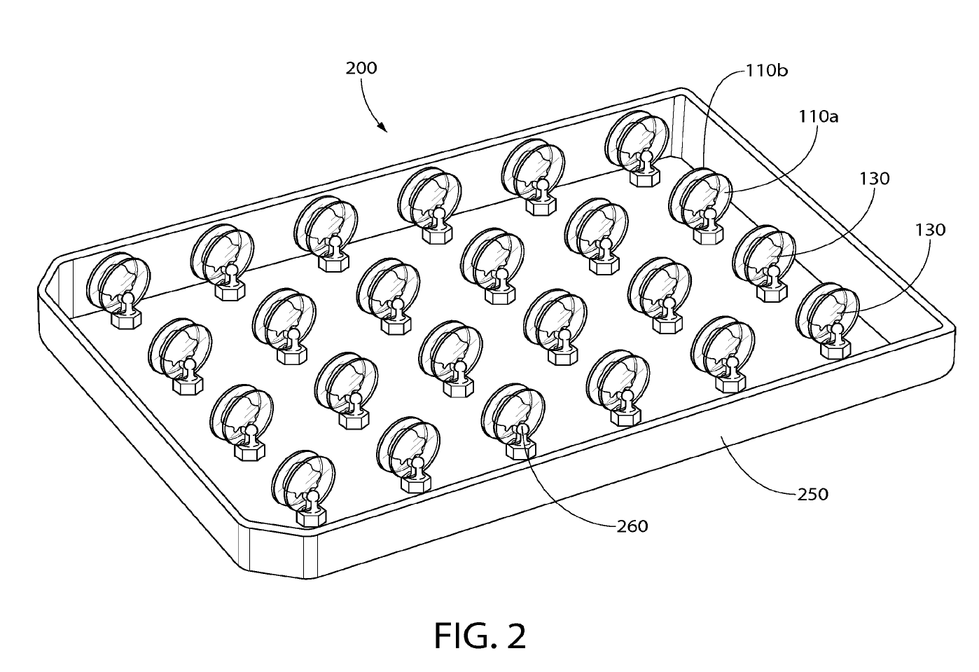

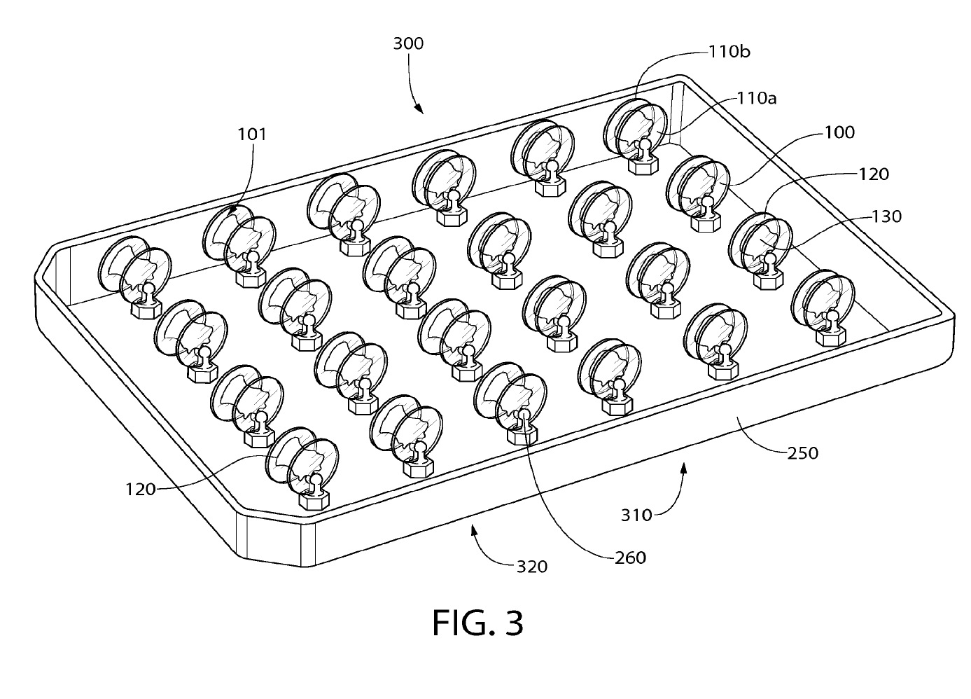

In some embodiments, the adhesive is heat-resistant and steam-resistant such that the in vitro oral biofilm model of the present disclosure, as depicted in FIG. 2 or FIG. 3, for example, can be autoclaved before or after use. Accordingly, in some embodiments, the adhesive is able to withstand high temperatures, for example, between about 120° C. and 150° C., at least about 124° C., at least about 134° C., at least about 136° C., at least about 140° C. In other embodiments, the adhesive is able to withstand temperatures greater than that generally required for autoclaving, e.g., greater than about 150° C., about 160° C., about 170° C., about 200° C., about 250° or about 300° C. In various embodiments, the adhesive material solidifies after adhering members of substratum pairs, such that there is no penetration of bacteria into the adhesive.

Biocompatible adhesives having the above-described characteristics are well known in the art and include, for example, which are commercially available from, e.g., Masterbond, Hackensack, N.J. In some embodiments, dental adhesives are used, e.g., polydimethylsiloxanecopolymers, including but not limited to adhesives used to adhere dental impressions to trays such as Coltene Adhesive AC, Coltene/Whaledent Inc., Altstätten, Switzerland. In other embodiments, polyvinylsiloxanes surface activated impression material is used, such as PRESIDENT® Plus Light Body, Coltene/Whaledent Inc.

In other embodiments, the separation element forms the space intervals between the first and second members without attaching the first and second members. For example, the space intervals between the members may be formed by a clamp.

Each substratum pair may be attached to a support using any means known in the art. For example, the substratum pair may be directly attached to a support using adhesives, such as biocompatible adhesives, e.g., dental adhesives. In some embodiments, a first member and a second member of a substratum pair are attached to a support via a clamp.

In some embodiments, more than one substratum pair is adhered to a support, such as at least 2, 4, 6, 12, 24, 36, 50, 60, 96, 384 or more substratum pairs. Accordingly, a support may contain a plurality of substratum pairs affixed thereto.

In embodiments, substrata utilized in the oral biofilm model consistent with embodiments of the present invention have surface areas ranging from about 100 mm² to about 3000 mm², typically ranging from about 100 mm² to about 2500 mm², more typically ranging from about 2200 mm² to about 500 mm², and still more typically ranging from about 500 mm² to about 390 mm². In some embodiments, the substrata are in the form of a circle and have a radius ranging from about 5 mm to about 12.5 mm. In some embodiments, the substrata are in the size and shape of a circular microscope coverslip, e.g. a circular microscope coverslip with a radius of about 6 mm.

In some embodiments, the thickness of the substrata ranges from about 0.13 mm to about 1.5 mm, more typically from about 0.1 mm to about 0.64 mm, even more typically from about 0.13 to about 0.25 mm, and even more typically from about 0.13 mm to 0.19 mm or about 0.13 to about 0.16 mm. In various embodiments, the thickness of each substratum of a substratum pair is the same. In other embodiments, the thickness of each substratum of a substratum pair is different.

The support to which the substratum pairs may be attached include a metal support, a glass support, a polystyrene support, a polyethylene support, a vinyl acetate support, a polypropylene support, a polymethacrylate support, a polyacrylate support, a polyethylene support, a polyethylene oxide support, a polysilicate support, a polycarbonate support, a polytetrafluoroethylene support, a fluorocarbon support, a nylon support, a silicon support, a rubber support, a polyanhydride support, a polyglycolic acid support, a polyhydroxyacid support, a polyester support, a polycaprolactone support, a polyhydroxybutyrate support, a polyphosphazene support, a polyorthoester support, a polyurethane support, and combinations thereof.

In some embodiments, after the substratum pairs have been fixed to the substrate, the substratum pairs are suspended within a vessel containing a liquid growth medium. The vessel may be designed to accommodate, e.g., 1, 2, 3, 4, 5, 6, 7, 8, 12, 24, 96 or 384 or more separate substratum pairs.

In some embodiments, the vessel which is used with the oral biofilm model includes a body having sides and a bottom defining the vessel. The body is adapted to receive the support and the affixed substratum pairs in a fluid tight communication, which is capable of retaining a liquid growth medium therein. Appropriate vessels include, for example, commercially available Petri dishes, a 4-, 6-, 8-, 12-, 24-, 96-, or 384-well plastic tissue plates or Petri dishes, e.g. a 2 cm², 24-well plate. In some embodiments, the vessel is chosen such that the vessel includes wells, which correspond to the number of substratum pairs attached to the lid.

Useful materials for the vessels include, but are not limited to, glass, polystyrene, polypropylene, polycarbonate, copolymers (e.g., ethylene vinylacetate copolymers) and the like.

In some embodiments, the support is a lid, which has a surrounding lip that fits tightly over a surrounding wall of a vessel. The lid may be disposed upon a vessel and a fluid tight seal is formed between the walls of the lid and the vessel. This fluid tight enclosure prevents contamination of the liquid growth medium disposed within the vessel.

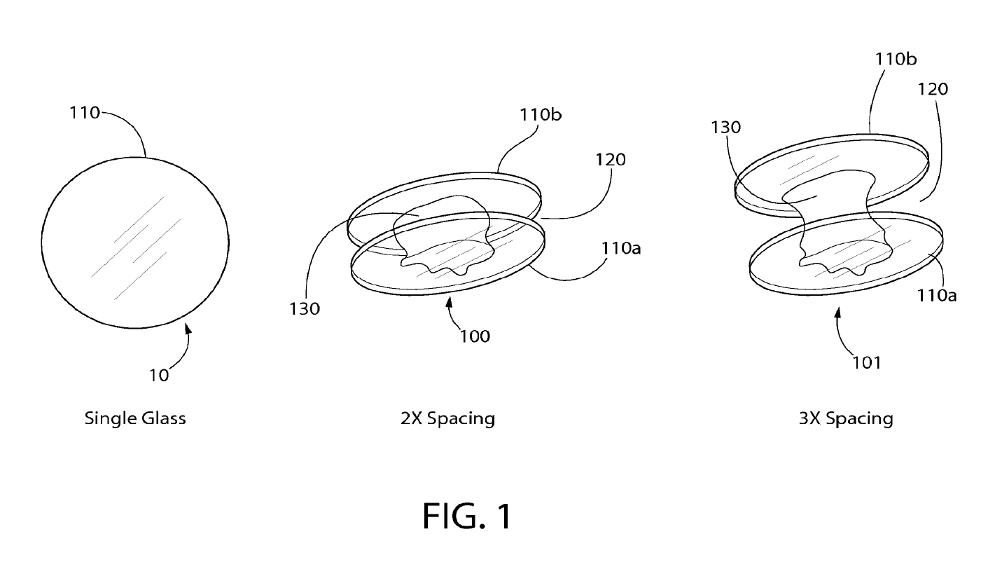

Referring now to FIG. 1, there is shown a view of a prior art substratum (10) consisting of a single glass slide (110). Examples of devices formed of substrata pairs of the oral biofilm model consistent with the examples of the present disclosure are shown in (100) and (101). Each substratum pair (100) and (101) includes a first member (110a) and a second member (110b). A separation element (130) separates the first member (110a) and the second member (110b) from each other, and in this embodiment, also attaches the first member (110a) and the second member (110b) to each other. The members of each substratum pair (100) and (101) are separated by a space interval (120). In this embodiment, the separation element (130) arranges a constant or uniform space interval between the members, as the major planes of the members are parallel to each other.

In some embodiments, the space interval (120) models interdental spacing. In FIG. 1, the space interval (120) of substratum pair (100) is smaller (2X) than the space interval (120) shown in embodiment (101), which is designated as 3λ. The space interval between a first and second member of a substratum pair may range from about 0.1 mm to about 10 mm, from about 1 mm to about 10 mm, from about 1 mm to about 5 mm, from about 1 mm to about 4 mm, from about 1 mm to about 3 mm, from about 1 mm, from about 2 mm, from about 3 mm, from about 4 mm, from about 5 mm, from about 6 mm, from about 7 mm, from about 8 mm, from about 9 mm and from about 10 mm. In some embodiments, the spacing or space interval (120) between the first and second member of the substratum pair may be uniform. In other embodiments, the spacing or space interval (120) may vary (not shown), for example from 0.5 mm to 2 mm, by, for example, setting the first member and the second member at an intersecting angle with each other.

FIG. 2 depicts an embodiment (200) of twenty-four substratum pairs (100) affixed to a support, e.g., in the form of a metal lid (250). The members (110a and 110b) of the substratum pairs (100) are affixed to a clamp (260) and the clamp (260) is affixed to the metal lid (250). In this embodiment, the clamps (260) are affixed to the metal lid using an adhesive (not shown). The separation element (130) between each member (110a and 100b) of the substratum pairs in this embodiment is an adhesive.

FIG. 3 shows an embodiment (300) of twenty-four substratum pairs (100 and 101) affixed to a metal lid support (250). Twelve of the substratum pairs (100) in the right portion (310) of the metal lid (250) have an interval (120) of 2× between the members (110a and 110b). Twelve of the substratum pairs (101) in the left portion (320) of the metal lid (250) have an interval (120) of 3λ.

The members (110a and 110b) of the substratum pairs (100) of FIG. 3 are affixed to a clamp (260) and the clamp (260) is affixed to the metal lid (250). In this embodiment, the clamps (260) are affixed to the metal lid using an adhesive (not shown). The separation element (130) between each member (110a and 100b) of the substratum pairs in this embodiment is an adhesive.

In some embodiments the substratum pairs attached to the support may be suspended in a liquid growth medium in a vessel (not shown), e.g. a 24-well plate, which allows biofilm to form on each member of a substratum pair.

The support device of the present disclosure allows the exposure time/growth time of the biofilm to be carefully monitored and controlled by removing the entire support from a vessel wherein all of the substratum pairs are affixed to the support. Therefore, the process of removing the support may correlate to removing all of the substratum pairs from a liquid growth media simultaneously. Thus, the support promotes uniform formation of biofilm on each of the substratum pairs because all of the substratum pairs may be removed from a vessel in a single action. The production of uniform biofilms may ensure that test results are uniform and accurate. Still further, the oral biofilm model of the present disclosure allows for high throughput of biofilm formation because a large number of substratum pairs may be prepared at once.

In some embodiments, the in vitro oral biofilm model of interdental spaces allows the evaluation of biofilm formation on substratum pairs having different space intervals (120) to be simultaneously tested in a liquid growth medium to assess the effects of varying intervals on biofilm formation. In other embodiments, the space between each substratum member of a substratum pair is the same for each of the substratum pairs on a support. In other embodiments, the material used to form a substratum pair may vary between substratum pairs on a support, e.g., some of the substratum pairs of the present oral biofilm model may be enamel while other substratum pairs may be composed of a different material, e.g. glass. Thus, the present oral biofilm model at least allows for testing the formation of biofilm on different materials and/or for testing the effect of different or varying distances (spaces) between the first and second substratum pair members.

### Methods of Using the In Vitro Oral Biofilm Model of Interdental Spacing

As noted above, the in vitro oral biofilm model of interdental spacing device may be used to grow biofilms and to assess the characteristics of the biofilms. For example, the effects of a particular interdental space, such as a 2 mm interval between members of a substratum pair, on biofilm formation may be assessed using the oral biofilm model of the present disclosure.

In some embodiments, biofilms are formed on the substratum pairs by incubating the substratum pairs in a vessel containing a liquid growth medium for a period of time to allow a biofilm to form on the substratum pairs, for example at 37° C. under anaerobic conditions, such as 10% CO₂, 10% H₂, and 80% N₂.

The period of time allowed for biofilm formation ranges from about 2 hours to about five days, about 3 hours to about 48 hours, about 4 hours to about 24 hours, about 16 hours or about 8 hours. In some embodiments, there may be two incubation periods. For example, there may be a first incubation period wherein the substratum pairs are incubated in a liquid growth medium containing microorganisms, which are capable of forming a biofilm, followed by a second incubation in the presence of a liquid growth medium, which does not contain biofilm-forming microorganisms. In various embodiments, the second incubation contains microorganisms. In some embodiments, the first and second incubations are repeated one, two, three or more times.

The first incubation time period may range from about 2 hours to about 24 hours, more typically from about 3 hours to about 12 hours, or more typically from about 4 hours to about 10 hours, or even more typically about 6 hours or about 8 hours.

The second incubation period may range from about 2 hours to about five days, from about 3 days to about 5 days, or more typically about 48 hours or about 24 hours or about 16 hours. In some embodiments, the second incubation may be from about 3 hours to about 12 hours, or about 4 hours to about 10 hours, or from about 6 hours or about 8 hours. After formation of a biofilm, the biofilm may be removed from the substratum pairs by sonication for example, to assess the biofilm formation.

Assessment of the biofilm formation may be determined by, for example, the use of confocal laser scanning microscopes to observe biofilm morphology and/or adherence to a substratum pair. The number of colony forming units in each of the formed biofilms may also be determined. Enumeration of bacteria present in the biofilms can also be achieved by using molecular approaches such as quantitative polymerase chain reaction (qPCR or Real-Time PCR). In some embodiments, biofilm formation is assessed by optical density measurement as an indicator of bacterial counts. In other embodiments, Resazurin fluorescence is used to assess the aerobic respiration of each biofilm as an indicator of bacterial counts.

The liquid growth medium, which may be used with the oral biofilm model and methods described herein may be any liquid growth medium known in the art for growing biofilms. For example, brain heart infusion medium containing 18.5 g/I brain heart infusion, 0.2% sucrose and 50 mmol/l PIPES at pH 7.0 may be used. In other embodiments, a (4:1) saliva-like medium (SLM, 0.1% Lab Lemco Powder, 0.2% yeast extract, 0.5% peptone, 0.25% mucine from porcine stomach, type III (Sigma-Aldrich), 6 mM NaCl, 2.7 mM KCl, 3.5 mM KH₂PO₄, 1.5 mM K₂HPO₄, 0.05% urea, pH 6.7) (1:3) is used. Alternatively, a chemically defined medium (CDM) may be used without any glucose or supplemented with human serum (4:1), 50 mM glucose or 50 mM sucrose is used, see Rijn and Kessler, Infect Immun., 1980, 27(2):444-448 incorporated herein by reference. In some embodiments, McBain medium is used, which contains, sucrose, hemin, vitamin K, and fresh or frozen saliva, see McBain et al., 2005, “Development and characterization of a simple perfused oral microcosm”, J. Appl. Microhiol, 98, 624-634, which is incorporated herein by reference. In some embodiments, the liquid growth medium comprises glucose or sucrose.

The in vitro oral biofilm model of the present disclosure is suitable for formation of biofilms caused by plaque-producing microorganisms and/or the formation of biofilms caused by microorganisms responsible for periodontal disease. In some embodiments, the model may be used for the formation of biofilms caused by periodontal disease-producing microorganisms.

In some embodiments, the liquid growth medium contains one or more biofilm forming organisms. In some embodiments, the biofilm forming microorganisms are those belonging to the genera, which are associated with periodontal disease, which include but are not limited to the Treponema, Bacteroides, Porphyromonas, Prevotella, Capnocytophaga, Peptostreptococcus, Fusobacterium, Actinobacillus, and Eikenella. In other embodiments, the liquid growth medium contains one or more periodontal associated species, such as Treponema denticola, Porphyromonas gingivalis, Bacteroides forsythus, Prevotella intermedia, Prevotella nigrescens, Pepostreptococcus micros, Fusobacterium nucleatum subspecies, Eubacterium nodatum or Streptococcus constellatus.

In other embodiments, the liquid growth medium contains at least one microorganism associated with dental plaque formation selected from the genera: Streptococcus, Veillonella, Actinomyces, Granulicatella, Lptotrichia, Lactobacillus, Thiomonas, Bifidobacterium, Propionibacterium or Atopobium. In other embodiments, the liquid growth medium contains one or more species associated with dental plaque formation including but not limited to Streptococcus mutans, Streptococcus sobrinus, Streptococcus gordonii, Streptococcus sanguinis, Lactobacillus acidophilus, Lactobacillus casei, Lactobacillus fermentum, Lactobacillus delbrueckii, Lactobacillus plantarum, Lactobacillus jensenii, Lactobacillus brevis, Lactobacillus salivarius Lactobacillus gasseri and Actinomyces naeslundii. In other embodiments, the liquid growth medium at least contains Streptococcus mutans.

In some embodiments, the liquid growth medium may contain saliva from a mammal, such as humans, non-human primates, camels, cats, chimpanzees, chinchillas, cows, dogs, goats, gorillas, horses, llamas, mice, pigs, murine, rats and sheep. In some embodiments, human saliva is used.

Using the oral biofilm model of the present disclosure, the effects of test agents on interdental spacing may be assessed. The test agents may be oral compositions, e.g. mouthwash or toothpaste. As demonstrated by the Examples, effects of test agents on interdental spacing intervals may not be recognized when using prior art models, such as those which use only a single substratum, e.g. a single glass for biofilm formation.

In some embodiments, identification of biofilm reducing agents may be assessed by incubating the substratum pairs of the oral biofilm model in a liquid growth medium containing at least one microorganism, which is capable of forming a biofilm or incubating a liquid growth medium with saliva. The substratum pairs may then be contacted with the test agent and any reduction in biofilm assessed.

For example, after an 8 hour initial biofilm formation, the substratum pairs may be contacted with a test agent. After this first treatment with a test agent (for example, a morning treatment), the biofilm is again incubated after removal of the test agent with fresh liquid growth medium (containing microorganisms) for about 8 hours, treated again with test agent (for example, an evening treatment) and after removal of the test agent, incubated again for 16 hours (overnight) in a liquid growth medium without microorganisms. The treatments and incubations may repeated, for example, for 3 more days (approximately 7 treatments total) to mimic consumer twice-a-day usage of the toothpaste/mouthwash over the course of a week.

The biofilm may then be removed from the substratum pairs by sonication and efficacy of the test agent may be determined. For example, a decrease in an amount of biofilm on a substratum pair after treatment with a test agent in comparison to an amount of biofilm formed on a substratum pair, which was not treated with the test agent, indicates that the test agent may be used to reduce biofilm accumulation.

In some embodiments, the effects of a test agent are assessed after a second incubation. For example, after an initial 8 hour biofilm formation, the substratum pairs of the oral biofilm model of the present agent are contacted with a test agent, followed by a second incubation at 37° C. under anaerobic conditions in a liquid growth medium in the absence of microorganisms. The second incubation may be for a time period as described above.

The test agents identified as described herein may be administered to a patient in need thereof. The patient may be a mammal, e.g., human, non-human primate, camel, cat, chimpanzee, chinchilla, cow, dog, goat, gorilla, horse, llama, mouse, pig, rat and sheep.

In addition to using the in vitro oral biofilm model of the present disclosure for identifying test agents which reduce biofilm formation, the use of the model may be readily expanded to identify agents, for example, which promote biofilm formation and/or which reduce or promote biofilm formation in combination with different spacing intervals between the substratum pairs. The oral biofilm model of this disclosure may also be used to test the efficacy of test agents on different biofilm producing microorganisms.

### EXAMPLES

### Example 1

Preparation of the In Vitro Biofilm Model of Interdental Spacing and Biofilm Formation

Two in vitro oral biofilm models of interdental spacing were prepared. The substratum pairs of each model were prepared by gluing glass disks (12 mm diameter glass coverslips) together at set intervals with dental glue to create two models of interdental space (a 2× interval model and a 3× interval model as shown in FIG. 1). PRESIDENT® Plus Light Body, (Coltène/Whaledent Inc., Altstätten, Switzerland) was used as the adhesive. Control substrata were prepared using only a single glass slide as described, for example, in Exterkate et al., “Different Response to Amine Fluoride by Streptococcus mutans and Polymicrobial Biofilms in a Novel High-Throughput Active Attachment Model”, Caries Res. 2010, 44:372-379, herein incorporated in its entirety by reference.

A total of 24 substratum pairs and controls were prepared for the 2× model and the 3× model. The 2× model was prepared according to FIG. 2 except that some of the substratum pairs of FIG. 2 were replaced with single glass slide controls. After assembling the lid and substratum pairs, each in vitro biofilm model was autoclaved. The lid for each model fit onto a standard polystyrene 24-well plate.

A liquid growth medium was prepared including of 2.5 g/l mucin, 2.0 g/l Bacto peptone, 2.0 g/l Trypticase peptone, 1.0 g/l yeast extract, 0.35 g/l NaCl, 0.2 g/l KCL, 0.2 g/l CaCl₂, 0.001 g/l hemin and 0.0002 g/l vitamin K1 as described in McBain et al., 2005, “Development and characterization of a simple perfused oral microcosm”, J. Appl. Microbiol, 98, 624-634, herein incorporated in its entirety by reference.

Saliva was collected on ice from a single donor. The saliva was diluted 2-fold with 60% sterile glycerol to protect the bacterial cells from cryodamage. Saliva was stored at −80° C.

1.5 milliliters of the liquid growth medium inoculated with saliva was added to each well of two polystyrene 24-well plates. The substrata of the in vitro oral biofilm models were incubated anaerobically (10% CO₂, 10% H₂ and 80% N₂) for up to 5 days at 37° C. to form a biofilm on each of the substratum pairs. Optical density at 610 nm was used to assess the physical material in each biofilm as an indicator of bacterial counts. Biofilm suspension absorption was measured on a Perkin Elmer (Waltham, Mass.) EnVision®, Multilabel Reader.

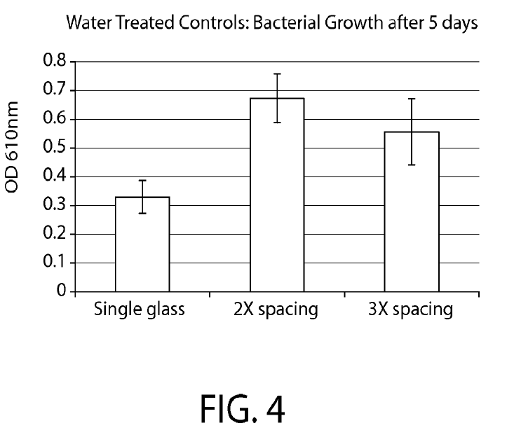

As depicted in Table 1 and FIG. 4, the bacteria on a single glass disk substratum as described in Exterkate et al. only grow to an OD of approximately 0.3. In contrast, the bacteria grow to an OD of approximately 0.67 using the 2× spacing model and approximately 0.55 using the 3× spacing model. Accordingly, the amount of bacteria, which grows on an oral biofilm model using the 2× or 3× oral biofilm model is approximately double the amount of that grown on a single substratum due to increased surface area.

TABLE 1

| | Ave | |
|---|---|---|
| Test Samples | OD | Std Dev |
| Single glass | 0.3295 | 0.057449 |
| 2X spacing | 0.67325 | 0.085367 |
| 3X spacing | 0.55675 | 0.114968 |

Effects of Test Agents on Biofilm Reduction

The oral biofilm models prepared as described above were also used to test the efficacy of oral compositions. After an initial biofilm attachment phase of 8 hours under anaerobic conditions at 37° C., each model was removed from the 24-well plate and the efficacy of toothpaste alone (Colgate Max Fresh) versus toothpaste and mouthwash (0.075% Cetylpyridinium chloride) on biofilm reduction was assessed. To begin with, the lid of each model was moved up and down 10 times in liquid growth medium without saliva to remove loose cells. Each lid was then transferred to a 24-well plate containing 1.6 milliliters of a 1:2 toothpaste slurry and then incubated for 2 minutes at room temperature. Water-containing wells were used as controls. Each lid was subsequently transferred to another new plate for washing with 1.7 milliliters Cysteine Peptone Water (CPW) and shaken for 5 minutes to wash away the treatment solutions. The wash procedure was performed twice, each time with fresh CPW in a 24-well plate.

The lid was then transferred to a 24-well plate containing 1.6 milliliters of mouth rinse solution and was incubated for 10 minutes at room temperature. Water-containing wells were used as controls. The lid was subsequently transferred to a new plate for washing with 1.7 milliliters of CPW and moved up and down 10 times to wash away the treatment solutions. The wash procedure was performed three times, each time with fresh CPW in a 24-well plate. The biofilms were transferred into growth medium without microorganisms and incubated anaerobically at 37° C. up to the next treatment exposure. There were 4 biofilm replicates for each test product (N=4).

The treatments in paragraphs [0065] and [0066] were repeated at 24 hours, 32 hours, 48 hours, 56 hours, 72 hours, 80 hours and 96 hours with incubations between each treatment of 8 hours or 16 hours.

Live/Dead ratios were also determined to quantify the percent of biofilm left viable after the last treatment. Biofilm suspension was incubated 1:1 with Invitrogen BacLight™ Live/Dead® viability kit using SYTO 9 green-fluorescent nucleic acid stain and red-fluorescent propidium iodide (Molecular Probes, Cat. No. L7012) for 15 minutes at room temperature in the dark. Fluorescence was read by exciting the samples at 485 nm and reading emission at 535 nm and 635 nm measured on a Perkin Elmer EnVision® Multilabel Reader.

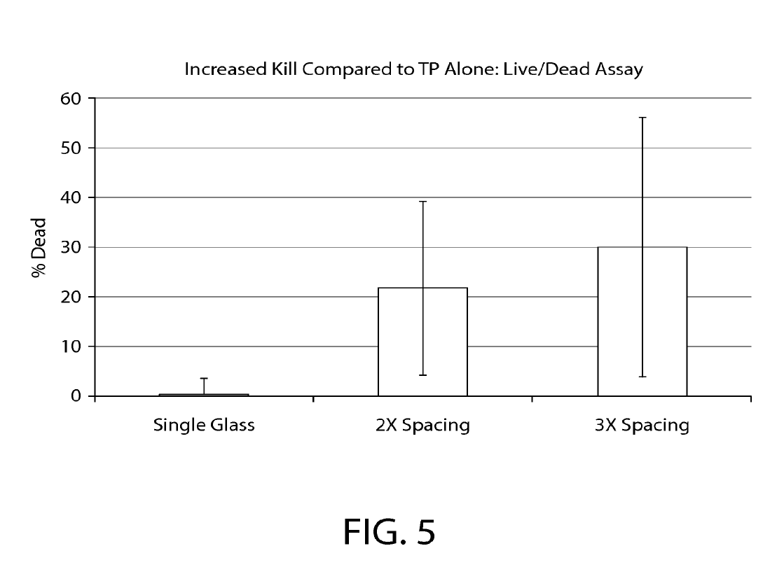

Table 2 and FIG. 5 show that the combination of toothpaste and mouthwash reduces the amount of live bacteria in a biofilm more than toothpaste alone using the in vitro oral biofilm interdental spacing model. There was no effect on the comparative biofilm reduction between the use of toothpaste only or toothpaste and mouthwash on the biofilm model using only a single glass slide as a substratum. Moreover, by increasing the space interval of the substratum pairs to 3× from 2λ, the amount of live bacteria was more greatly reduced. See Table 2.

TABLE 2

| | Average | |
|---|---|---|
| | Increased Kill | |
| Test Samples | (% dead) | Std Dev |
| Single Glass | 0.112805 | 3.464663 |
| 2X Spacing | 21.62938 | 17.64911 |
| 3X Spacing | 30.13085 | 26.16061 |

Resazurin fluorescence was used to assess the aerobic respiration of each biofilm as an indicator of bacterial counts after the last treatment. Biofilm suspensions were incubated 1:1 with resazurin dye and incubated at 37° C. for 3-5 minutes (or until pink color observed). Fluorescence was measured on a Perkin Elmer EnVision® Multilabel Reader.

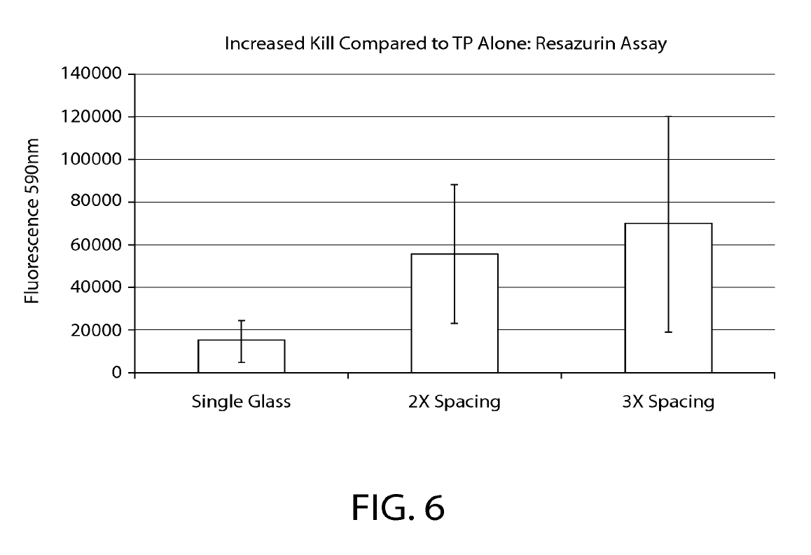

Table 3 and FIG. 6 also demonstrate that the in vitro oral biofilm interdental spacing model of the present disclosure may be used to detect effects of test agents, which are not apparent using a model with only a single glass slide as a substratum. As shown in FIG. 6, which describes the results of the resazurin assay, the combination of mouthwash and toothpaste is more efficacious for killing bacterial cells in interdental space regions than the toothpaste alone. The beneficial effects of the combination of toothpaste and mouthwash are not clearly evident from the in vitro biofilm model using only a single glass slide as the substratum. As shown in Table 3, a fluorescent reading at 590 nm of only 14279 was observed for the single glass slide in comparison to the readings for the oral biofilm model of the present disclosure, i.e., 54998.25 (2× spacing) and 68993.25 (3× spacing).

TABLE 3

| | Average | |
|---|---|---|
| | Increased | |
| | Kill | |
| Test | (Fluorescence | |
| Samples | 590 nm) | Std Dev |
| Single Glass | 14279 | 9650.16 |
| 2X Spacing | 54998.25 | 32513.12 |
| 3X Spacing | 68993.25 | 50497.02 |

What is claimed is:

1. An in vitro oral biofilm model of interdental spaces comprising:
a support, and
at least two substratum pairs attached to the support, wherein an oral biofilm is capable of forming on the substratum pairs, wherein the substratum pairs each comprise a first member and a second member, and
wherein the first member and the second member of each substratum pair are arranged to form a space interval between the first member and second member, and
wherein the space interval between the first and second member of a first substratum pair is greater than the space interval between the first and second member of a second substratum pair.

2. (canceled)

3. The in vitro oral biofilm model of claim 1, wherein the space interval between the first member and second member of the substratum pair is about 0.1 mm to about 4 mm.

4. The in vitro oral biofilm model of claim 1, wherein the support is a lid.

5. The in vitro oral biofilm model of claim 1, wherein the substratum pair is formed from a synthetic material.

6. The in vitro oral biofilm model of claim 5, wherein the synthetic material is selected from the group consisting of synthetic hydroxyapatite, glass and ceramic.

7. The in vitro oral biofilm model of claim 1, wherein the substratum pair is formed from a natural material.

8. The in vitro oral biofilm model of claim 7, wherein the natural material is enamel.

9. The in vitro oral biofilm model of claim 4, wherein the first member and the second member of the substratum pair are in the form of disks.

10. The in vitro oral biofilm model of claim 1, further comprising a vessel having sides and a bottom, said vessel adapted to receive said support and said substratum pairs, wherein said support is a lid and wherein the lid fits over the sides of the vessel.

11. The in vitro oral biofilm model of claim 1, further comprising:
a separation element, wherein the first member and the second member of the substratum pair are attached to each other by the separation element.

12. The in vitro oral biofilm model of claim 1, wherein the substratum pair is attached to the support by a clamp.

13. The in vitro oral biofilm model of claim 1, wherein a plurality of at least twenty-four substratum pairs is attached to the support.

14. A method of forming a biofilm using the model of claim 1.

15. A method of forming an oral biofilm, the method comprising:
providing a substratum pair attached to a support, wherein the substratum pair comprises a first member and a second member, and wherein the first member and the second member are separated to form a space interval between the first member and the second member, and wherein the space interval between the first and second member of a first substratum pair is greater than the space interval between the first and second member of a second substratum pair, and
providing a liquid growth medium comprising microorganisms capable of oral biofilm production;
placing the substratum pair into the liquid growth medium; and
incubating the substratum pair in the liquid growth medium to form a biofilm on the substratum pair.

16. The method of claim 15, wherein the substratum pair is formed from a natural material.

17. The method of claim 16, wherein the natural material is enamel.

18. The method of claim 15, wherein the substratum pair is formed from a synthetic material.

19. The method of claim 18, wherein the synthetic material is selected from the group consisting of synthetic hydroxyapatite, glass and ceramic.

20. The method of claim 15, wherein the incubating is under anaerobic conditions.

21. The method of claim 15, wherein the incubating has a time period from 3 hours to about 24 hours.

22. The method of claim 15, wherein the incubating has a time period of about 8 hours.

23. The method of claim 15, wherein the space interval between the first member and the second member of the substratum pair is about 0.1 mm to about 4 mm.

24. The method of claim 15, wherein the method further comprises:
placing the substratum pair into a liquid growth medium without microorganisms capable of oral biofilm production; and
incubating the substratum pair in the liquid growth medium without microorganisms capable of oral biofilm production for a time period ranging from 16 hours to five days.

25. The method of claim 15, wherein the liquid growth medium comprises saliva.

26. The method of claim 15, wherein the microorganisms are selected from at least one of the group consisting of Treponema denticola, Porphyromonas gingivalis, Bacteroides forsythus, Prevotella intermedia, Prevotella nigrescens, Peptostreptococcus micros, Fusobacterium nucleatum subspecies, Eubacterium nodatum, Streptococcus constellatus, Streptococcus mutans, Streptococcus sobrinus, S. gordonii, S. sanguinis, Lactobacillus acidophilus, Lactobacillus casei, Lactobacillus fermentum, Lactobacillus delbrueckii, Lactobacillus plantarum, Lactobacillus jensenii, Lactobacillus brevis, Lactobacillus salivarius Lactobacillus gasseri and Actinomyces naeslundii.

27. The method of claim 26, wherein the microorganism is Streptococcus mutans.

28. A method for identifying an agent for reducing biofilm in interdental spaces, the method comprising:
providing at least a first substratum pair and a second substratum pair attached to a support, wherein an oral biofilm is capable of forming on the substratum pairs,
wherein each substratum pair comprises a first member and a second member, and wherein the first member and the second member are separated to form a space interval between the first member and second member;
providing a liquid growth medium comprising microorganisms capable of oral biofilm production;
placing the at least first substratum pair and the second substratum pair into the liquid growth medium comprising microorganisms capable of oral biofilm production;
incubating the at least first substratum pair and the second substratum pair, thereby forming a biofilm on the at least first substratum pair and the second substratum pair;
contacting the first substratum pair with a test agent; and
comparing an amount of biofilm on the first substratum pair with an amount on the second substratum pair; and
indicating that the test agent reduces biofilm when the amount of biofilm on the first substratum pair is smaller in comparison to the amount of biofilm on the second substratum pair.

29. The method of claim 28, wherein the incubating has a time period of about 8 hours.

30. The method of claim 28, wherein the incubating is under anaerobic conditions.

31. The method of claim 28, wherein the microorganisms are selected from at least one of the group consisting of Treponema denticola, Porphyromonas gingivalis, Bacteroides forsythus, Prevotella intermedia, Prevotella nigrescens, Peptostreptococcus micros, Fusobacterium nucleatum subspecies, Eubacterium nodatum, Streptococcus constellatus, Streptococcus mutans, Streptococcus sobrinus, S. gordonii, S. sanguinis, Lactobacillus acidophilus, Lactobacillus casei, Lactobacillus fermentum, Lactobacillus delbrueckii, Lactobacillus plantarum, Lactobacillus jensenii, Lactobacillus brevis, Lactobacillus salivarius Lactobacillus gasseri and Actinomyces naeslundii.

32. The method of claim 28, further comprising:
administering the indicated test agent to a patient in need thereof to reduce biofilm formation.
